(19) United States
(12) Patent Application Publication
(10) Pub. No.: US 2025/0177491 A1
(43) Pub. Date: Jun. 5, 2025

(54) COMPLEX COACERVATES OF LACTOFERRIN AND OSTEOPONTIN

(71) Applicant: SOCIÉTÉ DES PRODUITS NESTLÉ S.A., Vevey (CH)

(72) Inventors: MARIE NOELLE HORCAJADA, Echenevex (FR); NICOLAS BONNET, Beaumont (FR); JONAS HAUSER, Lausanne (CH)

(21) Appl. No.: 18/840,745

(22) PCT Filed: Feb. 21, 2023
(86) PCT No.: PCT/EP2023/054306

§ 371 Date: Aug. 22, 2024

(30) Foreign Application Priority Data
Feb. 25, 2022 (EP) 22158718.1

(51) Int. Cl. A61K 38/40; A61K 9/00; A61K 38/19

(52) U.S. Cl. CPC A61K 38/40; A61K 9/0095; A61K 38/19

(57) ABSTRACT

The present invention relates to a complex coacervate comprising lactoferrin and osteopontin, processes of producing the same and composition comprising the same. Moreover, the present invention relates to complexes comprising lactoferrin and osteopontin in the treatment and/or prevention of metabolic diseases and/or inflammatory diseases. Moreover, the present invention relates to complexes comprising lactoferrin and osteopontin for promoting bone development, growth, strength and/or healing, or preventing and/or treating a bone disease.

### FIELD OF THE INVENTION

The present invention relates to a complex coacervate comprising lactoferrin and osteopontin, processes of producing the same and composition comprising the same. Moreover, the present invention relates to complexes comprising lactoferrin and osteopontin in the treatment and/or prevention of metabolic diseases and/or inflammatory diseases. Moreover, the present invention relates to complexes comprising lactoferrin and osteopontin for promoting bone development, growth, strength and/or healing, or preventing and/or treating a bone disease.

### BACKGROUND OF THE INVENTION

Lactoferrin (LF) and osteopontin (OPN) have been identified as having beneficial health benefits and thus it has been attempted to use these proteins in nutritional products or pharmaceutical products.

Lactoferrin (LF) is an iron-binding glycoprotein found in the milk of most mammalian species. This protein typically occurs in human milk at concentrations of ˜4.91 and 2.10 g/L in early- and mature-milk, respectively, while in bovine milk it is present at ˜10-fold lower concentrations. LF is a protein with associated biological functions including anti-microbial, anti-inflammatory and immunomodulatory effects. Several clinical studies in infant populations have linked LF with reductions in incidence of late onset sepsis and necrotizing enterocolitis

LF is known to bind to anionic proteins in form of soluble complexes. When lactoferrin is added during wet mixing of an infant formula blend, the proteins undergo unfolding denaturation and refolding under the effect of temperature and pH, resulting in protein instability and increases in viscosity. This phenomenon makes it very complicated to add lactoferrin in wet (i.e., before drying) in a product such as infant formula.

Therefore, it has been described to add lactoferrin into products such as infant formula in solid form, by dry-mixing after the base powder of the product has been produced. Such process, however, is challenging because lactoferrin added in solid form needs to be sterile. To achieve the required level of sterility, lactoferrin would need to be subjected to sterilization techniques. Most sterilization techniques involve the use of high heat, which may lead to denaturation of the lactoferrin. Other sterilization techniques, such as membrane filtration are available but may be costly and require specific equipment. Also, addition of the sterile lactoferrin in the final product requires specific and precise aseptic dosing equipment.

Osteopontin (OPN) is a minor, acidic, highly phosphorylated glycoprotein, also present at higher concentrations in human milk compared with bovine milk. It has been reported that average OPN concentrations in human milk, bovine milk and infant formula to be 138, 18, and 9 mg/L, respectively. Several biological functions have been attributed to OPN, including the ability to stimulate immunological, brain and intestinal development. OPN-supplementation of infant formulae has also been reported to lower the incidence of fever while altering plasma cytokine patterns, resulting in lower levels of pro-inflammatory TNF-α and higher levels of interleukin-2.

In order to overcome the issues with sterilization of lactoferrin, it would be highly desirable to develop combined forms of lactoferrin and osteopontin, allowing addition of them together with other ingredients of an infant formula in the wet mix and aseptic processing or spray-drying in the infant formula composition.

Moreover, it would be highly desirable to enhance the bioactivity and/or bioavailability of lactoferrin in particular in combination with osteopontin for application to a subject, in particular in metabolic diseases and/or inflammatory diseases.

In particular, it would be highly desirable to improve the bioactivity and/or bioavailability of lactoferrin, in particular in combination with osteopontin in a subject once digested, in particular for treatment and/or prevention of metabolic diseases and/or inflammatory diseases.

Further, lactoferrin can also promote bone growth. At physiological concentrations, lactoferrin potently stimulates the proliferation and differentiation of primary osteoblasts and also acts as a survival factor inhibiting apoptosis induced by serum withdrawal.

Lactoferrin also affects osteoclast formation and can potently inhibit osteoclastogenesis (Naot, D., et al., 2005. Clinical Medicine & Research, 3(2), pp. 93-101).

Studies have shown that osteopontin also plays a role in bone metabolism and homeostasis. Osteopontin is an important factor in neuron-mediated and endocrine-regulated bone mass, and is involved in biological activities such as proliferation, migration, and adhesion of several bone-related cells. Osteopontin has been demonstrated to be closely related to the occurrence and development of many bone-related diseases, including osteoporosis (Si, J., et al., 2020. Medical science monitor: international medical journal of experimental and clinical research, 26, pp. e919159-1).

It would be highly desirable to enhance the bioactivity and/or bioavailability of lactoferrin and/or osteopontin to promote bone metabolism and/or homeostasis.

Up to this date, attempts to form coacervates of lactoferrin and osteopontin have been shown to be not successful.

### SUMMARY OF THE INVENTION

The present invention relates to a complex coacervate comprising lactoferrin and osteopontin.

The present invention also relates to a process of producing a complex coacervate according to any of the preceding claims, wherein the process comprises the steps of:
a. providing individual aqueous solutions comprising lactoferrin and osteopontin,
b. mixing the individual aqueous solutions comprising lactoferrin and osteopontin at a pH of 4 to 6, preferably at a pH of 4.5 to 5.5, more preferably at a pH of 4.8 to 5.2, even more preferably at a pH of 5 and wherein the individual aqueous solutions comprising lactoferrin and osteopontin are adjusted in that the protein mass ratio of lactoferrin to osteopontin is in the range of 2 to 8, preferably in the range of 3 to 6, more preferably in the range of 3.2 to 5.5, more preferably in the range of 3.5 to 5, even more preferably 3.8 to 4.2 and even more preferably 4.

The present invention also relates to a composition comprising the complex coacervate according to the present invention.

The present invention also relates to a complex, preferably a complex coacervate, comprising lactoferrin and osteopontin or a composition comprising a complex, preferably a complex coacervate, comprising lactoferrin and osteopontin for use in the treatment or prevention of metabolic disorders, in particular overweightness, obesity, pre-diabetes or diabetes, and/or inflammatory diseases, in particular sepsis or necrotizing enterocolitis.

The present invention also relates to a method for treating or preventing metabolic disorders, in particular overweightness, obesity, pre-diabetes or diabetes, and/or inflammatory diseases, in particular sepsis or necrotizing enterocolitis, by administering a complex, preferably a complex coacervate, comprising lactoferrin and osteopontin or a composition comprising a complex, preferably a complex coacervate, comprising lactoferrin and osteopontin to a subject.

The present invention also relates to a complex coacervate comprising lactoferrin and osteopontin for use in promoting bone development, growth, strength and/or healing, or preventing and/or treating a bone disease.

The present invention also relates to a method of promoting bone development, growth, strength and/or healing, or preventing and/or treating a bone disease, the method comprising administering to a subject in need thereof a therapeutically effective amount of a complex coacervate comprising lactoferrin and osteopontin.

### BRIEF DESCRIPTION OF THE DRAWINGS

Additional features and advantages of the present invention are described in, and will be apparent from, the description of the presently preferred embodiments which are set out below with reference to the drawings in which:

FIG. 1: Optical microscopy images (40× magnification) of lactoferrin (LF)-osteopontin (OPN) complex coacervates (prepared at pH 5, 5% w/v protein, LF:OPN mass protein ratio of 4:1) which were isolated and dispersed in ultrapure water to confirm the presence of spherical, liquid coacervates and the absence of irregular, solid co-precipitates. Panels A, B: phase contrast microscopy; Panel C: darkfield microscopy. All images were captured using a LabCam® smartphone microscope adapter.

FIG. 2: Differential scanning calorimetry thermograms of 5% w/v solutions of lactoferrin (dashed line) and osteopontin (dotted line) at pH 5.0 (FIG. 1, above) and LF/OPN complex coacervate at pH 5.0 (prepared at protein mass ratio 4:1, 5% w/v protein) which had a protein concentration of 27.4% (w/w).

FIG. 3: The impact of adding gastrointestinal digestates of lactoferrin (LF), osteopontin (OPN), LF-OPN soluble complexes (SC) or LF-OPN complex coacervates (CC) to a model of intestinal cell inflammation. All samples are added at 0.35 mg protein equivalent/mL. The inflammatory model used is Escherichia coli O111:B4 lipopolysaccharide (LPS)-induced NF-κB activation in HT-29 clone 34 cells. LPS (20 ng/mL) and human milk serum (5% v/v) are added to all wells. All data are normalized to give 100 relative luminescence units (RLU) for the LPS treatment and represent mean values±standard error of triplicate measurements from three independent experiments. ‘*’ represents statistically significant differences (P<0.05) from the 100% RLU treatment.

FIG. 4: The impact of lactoferrin-osteopontin soluble complex (SOLUBLE), lactoferrin-osteopontin coacervate complex (COACERVATE), and lactoferrin-osteopontin blend (BLEND) on bone development, growth and strength. C57/bl6 wild type mouse were supplemented orally between post-natal day 2 and 28 with three different osteopontin-lactoferrin mixes (soluble, blend or co-acervate complex, n=10 per group). From days 28 to 170 all mice received the same amount of a standard diet. At the end of the study femurs were collected to evaluate the following bone microstructure parameters: (A) trabecular bone volume and tissue volume fraction (BV/TV, %); (B) trabecular bone mineral density (Tb.BMD, mg HA/ccm); (C) cortical bone volume (Ct.BV, mm³); (D) medio-lateral diameter (ML diameter, mm); (E) antero-posterior diameter (AP diameter, mm); (F) force yield (N); and (G) stiffness (N/mm). (H) and (I) show exemplary trabecular and cortical structures obtained by micro-CT, respectively. (J) shows the directions for the medio-lateral and antero-posterior diameters.

### DETAILED DESCRIPTION OF THE INVENTION

### Definitions

As used herein, the following terms have the following meanings.

The term “complex coacervate” is well defined in the art. A complex coacervate is understood as a spherical droplet composed of at least two different assorted proteins which are primarily held together by electrostatic forces from a surrounding aqueous liquid. Cooper et al. [Current Opinion in Colloid and Interface Science, (2005), 10, 52-78] are defining complex coacervation by the separation of a macromolecular solution, composed of at least two macromolecules (typically oppositely charged polyelectrolytes), into two immiscible liquid phases. In this case, the complex coacervate is defined as either the macroscopic phase concentrated in macromolecules obtained after associative phase separation or the liquid droplets concentrated in macromolecules obtained after mixing the two dispersions containing oppositely charged macromolecules, i.e., proteins in this specific case. Thermodynamically, complex coacervates are formed by the aggregation of macromolecular complexes that are formed between two oppositely charges macromolecules (protein or polysaccharide) in order to reduce the free energy of the mixture as pointed out by Schmitt et al. [Handbook of Hydrocolloids, Second Edition. Woodhead Publishing, 2009, pp. 420-476]. Generally, macromolecular complexes and subsequent formation of coacervates are mediated by electrostatic interactions, and formation of coacervates occurs when aggregates of macromolecular complexes are reaching the electrostatic limit of colloidal stability, i.e., the surface ζ-potential is between −15 and +15 mV. Complex coacervates are different to soluble complexes.

The term “infant” means a child under the age of 12 months.

The term “young child” means a child aged between one and seven years. The expression “nutritional composition” means a composition which nourishes a subject. This nutritional composition is usually to be taken orally or intravenously, and it usually includes a lipid or fat source and a protein source.

In a particular embodiment, the composition of the present invention is a “synthetic nutritional composition”. The expression “synthetic nutritional composition” means a mixture obtained by chemical and/or biological means, which can be chemically identical to the mixture naturally occurring in mammalian milks (i.e., the synthetic composition is not breast milk).

The expression “infant formula” or IF as used herein refers to a foodstuff intended for particular nutritional use by infants during the first months of life and satisfying by itself the nutritional requirements of this category of person (Article 2(c) of the European Commission Directive 91/321/EEC 2006/141/EC of 22 Dec. 2006 on infant formulae and follow-on formulae). It also refers to a nutritional composition intended for infants and as defined in Codex Alimentarius (Codex STAN 72-1981) and Infant Specialities (incl. Food for Special Medical Purpose). The expression “infant formula” encompasses both “starter infant formula” and “follow-up formula” or “follow-on formula”.

A “follow-up formula” or “follow-on formula” is given from the 6th month onwards. It constitutes the principal liquid element in the progressively diversified diet of this category of person. The expression “baby food” means a foodstuff intended for particular nutritional use by infants or young children during the first years of life. The expression “infant cereal composition” means a foodstuff intended for particular nutritional use by infants or young children during the first years of life.

The term “fortifier” refers to liquid or solid nutritional compositions suitable for mixing with breast milk or infant formula.

The term “probiotic” means live microorganisms that, when administered in adequate amounts, confer a health benefit on the host (FAO/WHO, 2002). The microbial cells are generally bacteria or yeasts. All percentages are by weight unless otherwise stated.

### Process for Producing Complexes

### Complex Coacervates

The present invention relates to a complex coacervate comprising lactoferrin and osteopontin.

In a particular embodiment, the complex coacervate may comprise protein in an amount of not more than 50% w/w. In a particular embodiment, the complex coacervate may preferably comprise protein in an amount of not more than 40% w/w. In a particular embodiment, the complex coacervate may more preferably comprise protein in an amount of not more than 35% w/w.

In a particular embodiment, the complex coacervate may comprise protein in an amount of at least 5% w/w. In a particular embodiment, the complex coacervate may preferably comprise protein in an amount of at least 15% w/w. In a particular embodiment, the complex coacervate may more preferably comprise protein in an amount of at least 25% w/w.

In a particular embodiment, the complex coacervate may comprise protein in an amount of 5 to 50% w/w. In a particular embodiment, the complex coacervate may preferably comprise protein in an amount of 15 to 40% w/w. In a particular embodiment, the complex coacervate may more preferably comprise protein in an amount of 25 to 35% w/w.

The amount of protein in the complex coacervate can be determined using the method according to AOAC 991.20-1994.

In a particular embodiment, the complex coacervate may comprise water in an amount of not more than 95% w/w. In a particular embodiment, the complex coacervate may comprise water in an amount of not more than 85% w/w. In a particular embodiment, the complex coacervate may even more preferably comprise water in an amount of not more than 75% w/w.

In a particular embodiment, the complex coacervate may comprise water in an amount of at least 50% w/w. In a particular embodiment, the complex coacervate may comprise water in an amount of at least 60% w/w. In a particular embodiment, the complex coacervate may even more preferably comprise water in an amount of at least 65% w/w.

In a particular embodiment, the complex coacervate may comprise water in an amount of 50 to 95% w/w. In a particular embodiment, the complex coacervate may comprise water in an amount of 60 to 85% w/w. In a particular embodiment, the complex coacervate may even more preferably comprise water in an amount of 65 to 75% w/w.

The amount of water and protein in the complex coacervate can be determined using the method according to ISO 5537:2004.

In a particular embodiment, the complex coacervate has a particle size of at least 500 nm in the shortest dimension. In a particular embodiment, the complex coacervate has preferably a diameter of at least 600 nm in the shortest dimension. In a particular embodiment, the complex coacervate has more preferably a diameter of at least 700 nm in the shortest dimension. In a particular embodiment, the complex coacervate has more preferably a diameter of at least 900 nm in the shortest dimension.

The diameter of the complex coacervate can be determined using dynamic light scattering or by microscopy.

In a particular embodiment, the protein mass ratio of lactoferrin to osteopontin in the complex coacervate is not more than 8. In a particular embodiment, the protein mass ratio of lactoferrin to osteopontin in the complex coacervate is preferably not more than 6. In a particular embodiment, the protein mass ratio of lactoferrin to osteopontin in the complex coacervate is more preferably not more than 5.5. In a particular embodiment, the protein mass ratio of lactoferrin to osteopontin in the complex coacervate is more preferably not more than 5. In a particular embodiment, the protein mass ratio of lactoferrin to osteopontin in the complex coacervate is even more preferably not more than 4.2.

In a particular embodiment, the protein mass ratio of lactoferrin to osteopontin in the complex coacervate is at least 2. In a particular embodiment, the protein mass ratio of lactoferrin to osteopontin in the complex coacervate is preferably at least 3. In a particular embodiment, the protein mass ratio of lactoferrin to osteopontin in the complex coacervate is more preferably at least 3.2. In a particular embodiment, the protein mass ratio of lactoferrin to osteopontin in the complex coacervate is more preferably at least 3.5. In a particular embodiment, the protein mass ratio of lactoferrin to osteopontin in the complex coacervate is even more preferably at least 3.8.

In a particular embodiment, the protein mass ratio of lactoferrin to osteopontin in the complex coacervate is in the range of 2 to 8. In a particular embodiment, the protein mass ratio of lactoferrin to osteopontin in the complex coacervate is preferably in the range of 3 to 6. In a particular embodiment, the protein mass ratio of lactoferrin to osteopontin in the complex coacervate is more preferably in the range of 3.2 to 5.5. In a particular embodiment, the protein mass ratio of lactoferrin to osteopontin in the complex coacervate is more preferably in the range of 3.5 to 5. In a particular embodiment, the protein mass ratio of lactoferrin to osteopontin in the complex coacervate is even more preferably in the range of 3.8 to 4.2. In a particular embodiment, the protein mass ratio of lactoferrin to osteopontin in the complex coacervate is even more preferably 4.

In a particular embodiment, the complex coacervates has a zeta potential in the range of −15 and +15 mV. In a particular embodiment, the complex coacervates may preferably have a zeta potential in the range of −10 and +10 mV. In a particular embodiment, the complex coacervates may preferably have a zeta potential in the range of −8 to +5 mV.

The zeta potential can be measured using a Malvern Zetasizer Nano-ZS (Malvern Instruments Inc., Worchestershire, UK) equipped with Malvern Zetasizer software 7.02.

### Process of Production

The present invention also relates to a process of producing a complex coacervate according to the present invention, wherein the process comprises the steps of:
a. providing individual aqueous solutions comprising lactoferrin and osteopontin,
b. mixing the individual aqueous solutions comprising lactoferrin and osteopontin at a pH of 4 to 6, preferably at a pH of 4.5 to 5.5, more preferably at a pH of 4.8 to 5.2, even more preferably at a pH of 5 and wherein the individual aqueous solutions comprising lactoferrin and osteopontin are adjusted in that the protein mass ratio of lactoferrin to osteopontin is in the range of 2 to 8, preferably in the range of 3 to 6, more preferably in the range of 3.2 to 5.5, more preferably in the range of 3.5 to 5, even more preferably 3.8 to 4.2 and even more preferably 4.

According to the present invention, in step a. individual aqueous solutions comprising lactoferrin and osteopontin are provided. Thereby it is understood that an individual aqueous solution comprising lactoferrin and an individual solution of osteopontin is prepared and provided.

In a particular embodiment, the individual aqueous solutions comprising lactoferrin and osteopontin are adjusted to a particular pH before mixing. In a particular embodiment, the individual solutions comprising lactoferrin and osteopontin are adjusted to a pH of 4 to 6, preferably to a pH of 4.5 to 5.5, more preferably to a pH of 4.8 to 5.2, even more preferably to a pH of 5.

In an alternative particular embodiment, the individual aqueous solutions comprising lactoferrin and osteopontin are not adjusted to a particular pH before mixing.

According to the present invention, in step b. the individual aqueous solutions comprising lactoferrin and osteopontin are mixed at a pH of 4 to 6. Thereby it is understood that the individual aqueous solutions comprising lactoferrin and osteopontin are mixed and adjusted to a pH of 4 to 6 by the addition of an acid, preferably aqueous solution of HCl, or base, preferably aqueous solution of NaOH.

In a particular embodiment, the individual aqueous solutions comprising lactoferrin and osteopontin are mixed at a pH of 4.5 to 5.5. In a particular embodiment, the individual aqueous solutions comprising lactoferrin and osteopontin are preferably mixed at a pH of 4.8 to 5.2. In a particular embodiment, the individual aqueous solutions comprising lactoferrin and osteopontin are mixed even more preferably at a pH of 5.

According to the present invention, in step b. the individual aqueous solutions comprising lactoferrin and osteopontin are adjusted in that the protein mass ratio of lactoferrin to osteopontin in the mixed solution is in the range of 2 to 8.

In a particular embodiment, the individual aqueous solutions comprising lactoferrin and osteopontin are preferably adjusted in that the protein mass ratio in the mixed solution is in the range of 3 to 6. In a particular embodiment, the individual aqueous solutions comprising lactoferrin and osteopontin are more preferably adjusted in that the protein mass ratio in the mixed solution is in the range of 3.2 to 5.5. In a particular embodiment, the individual aqueous solutions comprising lactoferrin and osteopontin are more preferably adjusted in that the protein mass ratio in the mixed solution is in the range of 3.5 to 5. In a particular embodiment, the individual aqueous solutions comprising lactoferrin and osteopontin are even more preferably adjusted in that the protein mass ratio in the mixed solution is in the range of 3.8 to 4.2. In a particular embodiment, the individual aqueous solutions comprising lactoferrin and osteopontin are even more preferably adjusted in that the protein mass ratio in the mixed solution is in the range of 4.

In a particular embodiment, in step b. the ionic strength in the mixed aqueous solution is not higher than 30 mM added salts. Thereby it is understood that the mixed aqueous solution does not comprise more than 30 mM added salts, i.e. added salts that are comprised in the mixed solution in total and which in particular could have been added when providing the individual solutions of lactoferrin and osteopontin and/or during step b. when mixing the individual solutions of lactoferrin and osteopontin. Salts are thereby understood as not being positively and/or negatively charged lactoferrin and/or osteopontin. In a particular embodiment, salts are understood as inorganic salts. In a particular embodiment, salt is understood as NaCl.

In a particular embodiment, the ionic strength in the mixed aqueous solution is preferably not higher than 20 mM salts. In a particular embodiment, the ionic strength in the mixed aqueous solution is more preferably not higher than 10 mM salts. In a particular embodiment, the ionic strength in the mixed aqueous solution is more preferably not higher than 5 mM. In a particular embodiment, the ionic strength in the mixed aqueous solution is even more preferably not higher than 0.2 mM salts.

In a particular embodiment, in step b. the individual aqueous solutions comprising lactoferrin and osteopontin are adjusted in that the total protein concentration in the mixed solution is below the point of self-suppression.

In a particular embodiment, the individual aqueous solutions comprising lactoferrin and osteopontin are preferably adjusted in that the total protein concentration in the mixed solution is less than 8% w/v. In a particular embodiment, the individual aqueous solutions comprising lactoferrin and osteopontin are preferably adjusted in that the total protein concentration in the mixed solution is preferably less than 6% w/v.

In a particular embodiment, the individual aqueous solutions comprising lactoferrin and osteopontin are preferably adjusted in that the total protein concentration in the mixed solution is more than 2% w/v. In a particular embodiment, the individual aqueous solutions comprising lactoferrin and osteopontin are preferably adjusted in that the total protein concentration in the mixed solution is preferably more than 4% w/v.

In a particular embodiment, the individual aqueous solutions comprising lactoferrin and osteopontin are preferably adjusted in that the total protein concentration in the mixed solution are in the range of 2 to 8% w/v. In a particular embodiment, the individual aqueous solutions comprising lactoferrin and osteopontin are preferably adjusted in that the total protein concentration in the mixed solution is in the range of preferably 4 to 6% w/v.

In a particular embodiment, the complex coacervate formed in the process according to the present invention can be isolated by any method known to a skilled person.

In a particular embodiment, the complex coacervate may be subjected to a drying step, such as spray-drying, belt drying, tumble drying and/or freeze-drying.

### Composition Comprising the Complex Coacervates

The present invention also relates to a composition comprising the complex coacervate according to the present invention.

In a particular embodiment, the composition may comprise the complex coacervate according to the present invention and soluble complexes of lactoferrin and osteopontin. In a particular embodiment, the composition comprises the complex coacervate according to the present invention and soluble complexes of lactoferrin and osteopontin obtained from the process of preparing the complex coacervates according to the present invention.

The composition can be any type of composition in which the complexes can be incorporated, such as a composition in the form of a food or beverage product, an animal feed product, a nutritional supplement for human or animal, a pharmaceutical composition or a cosmetic composition. The product may be in solid, liquid or semi-liquid form.

Food and beverage products include all products intended to be consumed orally by human beings, for the purpose of providing nutrition and/or pleasure. It can for example be a nutritional composition, such as for infants and/or young children, for a pregnant or lactating woman or a woman desiring to get pregnant, for individuals in need of a special nutrition due to an adverse health condition or for elderly people. More preferably, the nutritional composition is selected from infant formula, infant cereals, follow-up formula, growing-up milks and milk products for pregnant and lactating women or for women desiring to get pregnant. Other examples of food and beverage products include dairy products such as milk products or yogurts, soups, sauces, sweet and savoury snacks, powdered drinks and cereal products.

The product can also be in the form of an animal food product or a nutritional supplement for animals. Preferably, the animal is a mammal. Examples of animals include primates (e.g., humans), cows, sheep, goats, horses, dogs, cats, rabbits, rats, mice, fish, birds and the like.

Nutritional supplements are typically present in the form of a liquid, a gel, a powder or a tablet or capsule. Powder supplements typically encompass supplements to be dissolved in water or to be sprinkled on food or in a beverage. Such supplements are intended to provide additional nutrients and/or a health benefit to the subject consuming it, as well as other beneficial ingredients, such as for example lactoferrin, lactadherin and/or lysozyme. A supplement according to the present invention can be used for providing nutrients and/or a health benefit to human beings, as well as to animals, as defined above. Nutritional supplements include for example powder supplements to be added to breast milk, for example for premature or low birth weight infants. It also includes supplements for pregnant or lactating woman or for woman desiring to get pregnant.

Pharmaceutical products include for example drops, syrups, powder, tablet or capsule products intended to treat of prevent an adverse medical condition in a subject in need thereof.

Cosmetic compositions are typically intended for an aesthetic effect on the body and may be for topical use or may be administered by oral route.

The composition of the present invention preferably comprises the complex coacervate of the present invention in a therapeutically effective amount.

In a preferred embodiment, the composition of the present invention is an infant formula, a starter infant formula, a follow-on formula, a baby food, an infant cereal composition, a growing-up milk, a fortifier such as a human milk fortifier, or a supplement. In such compositions, the complex coacervates of the present invention are preferably present in an amount providing from 0.001 to 3 g, preferably 0.01 to 2 g, more preferably 0.1 to 1 g of lactoferrin per litre of the composition.

All types of compositions according to the invention can be formulated and manufactured in accordance with the knowledge of the person skilled in the art. The complex coacervates of the present invention are advantageously robust enough to be processed together with the other ingredients of the composition.

For example, in the manufacture of a spray-dried product, such as an infant formula, a growing-up milk or a follow-up formula in powder form, the complex coacervates of the present invention are robust enough to be added in the wet mix and spray-dried together with the other ingredients of the product. This is advantageous from a process economy point of view, as aseptic dosing and dry mixing of sensitive proteins like lactoferrin is not needed. In addition, incorporation of the complexes in the wet mix is advantageous from a product structure point of view. In particular, the complex coacervates of the present invention will be admixed in a homogeneous way with the other ingredients. In contrast, dry mixed powders may lead to inhomogeneous products due difference in the properties of the admixed powders such as for example difference in density or particle size. Inhomogeneity may lead to inaccurate dosage of the proteins.

Therefore, a process of making a composition selected from an infant formula, a follow-up formula or a growing-up milk in powder form comprising preparing a product concentrate comprising complex coacervates of the present invention and spray-drying said product concentrate is also an object of the present invention. Preparing the wet mix and spray-drying are carried out in accordance with the general knowledge of the person skilled in the art.

The invention also provides a process for making a composition selected from a liquid, an infant formula, a starter infant formula, a follow-on formula, a baby food, an infant cereal composition, a growing-up milk, a fortifier such as a human milk fortifier, or a supplement comprising admixing the complexes of the present invention to the liquid product base and aseptically processing the product base comprising the complexes. The preparation of the liquid product base and the aseptic processing are carried out in accordance with the general knowledge of the person skilled in the art.

The composition of the invention can further comprise at least one probiotic (or probiotic strain), such as a probiotic bacterial strain.

The probiotic microorganisms most commonly used are principally bacteria and yeasts of the following genera: Lactobacillus spp., Streptococcus spp., Enterococcus spp., Bifidobacterium spp. and Saccharomyces spp. In some particular embodiments, the probiotic is a probiotic bacterial strain. In some specific embodiments, it is particularly Bifidobacteria and/or Lactobacilli.

Suitable probiotic bacterial strains include Lactobacillus rhamnosus ATCC 53103 available from Valio Oy of Finland under the trademark LGG, Lactobacillus rhamnosus CGMCC 1.3724, Lactobacillus paracasei CNCM 1-21 16, Lactobacillus johnsonii CNCM 1-1225, Streptococcus salivarius DSM 13084 sold by BLIS Technologies Limited of New Zealand under the designation K12, Bifidobacterium lactis CNCM 1-3446 sold inter alia by the Christian Hansen company of Denmark under the trademark Bb 12, Bifidobacterium longum ATCC BAA-999 sold by Morinaga Milk Industry Co. Ltd. of Japan under the trademark BB536, Bifidobacterium breve sold by Danisco under the trademark Bb-03, Bifidobacterium breve sold by Morinaga under the trade mark M-16V, Bifidobacterium infantis sold by Procter & Gamble Co. under the trademark Bifantis and Bifidobacterium breve sold by Institut Rosell (Lallemand) under the trademark R0070. The composition according to the invention typically contains from 10e3 to 10e12 cfu of probiotic strain, more preferably between 10e7 and 10e12 cfu of probiotic strain per g of composition on a dry weight basis. In one embodiment the probiotics are viable. In another embodiment the probiotics are non-replicating or inactivated. There may be both viable probiotics and inactivated probiotics in some other embodiments.

The composition of the invention can further comprise at least one non-digestible oligosaccharide (e.g. prebiotics) other than the human milk oligosaccharides previously mentioned. They are usually in an amount between 0.3 and 10% by weight of composition. Prebiotics are usually non-digestible in the sense that they are not broken down and absorbed in the stomach or small intestine and thus remain intact when they pass into the colon where they are selectively fermented by the beneficial bacteria. Examples of prebiotics include certain oligosaccharides, such a fructooligosaccharides (FOS) and galactooligosaccharides (GOS). A combination of prebiotics may be used such as 90% GOS with 10% short chain fructo-oligosaccharides such as in the product by BENEO-Orafti sold under the trademark Orafti® oligofructose (previously Raftilose®) or 10% inulin such as in the product sold by BENEO-Orafti under the trademark Orafti® inulin (previously Raftiline®). A particularly preferred combination of prebiotics is 70% short chain fructo-oligosaccharides and 30% inulin, which is a product sold by BENEO-Orafti under the trademark “Prebio 1”.

The composition of the invention can further comprise at least one phage (bacteriophage) or a mixture of phages, preferably directed against pathogenic Streptococci, Haemophilus, Moraxella and Staphylococci. The composition according to the invention can be a nutritional composition, a preparation or a food product.

The composition according to the invention can be for example a nutritional composition such as a synthetic nutritional composition. It can be an infant formula, a starter infant formula, a follow-on formula, a baby food, an infant cereal composition, a growing-up milk, a fortifier such as a human milk fortifier, or a supplement.

When the composition is a supplement, it can be provided in the form of unit doses. In some embodiments the composition of the present invention is typically an infant formula. The composition of the present invention is typically used in infants or young children who were born by C-section.

These infants and young children represent a specific group of subjects requiring particular needs and care and the present inventors have surprisingly found that a composition comprising at least one human milk oligosaccharides and/or a precursor thereof is particularly effective for use in decreasing the incidence of necrotizing enterocolitis in these infants born by C-section.

The composition according to the invention can be used in term or preterm infants born by C-section.

Advantageously the composition of the invention is for use in termed infants or preterm infants in particular born by C-section.

In some embodiments the composition of the invention is for use in infants who are small for gestational age and born by C-section. In some embodiments the composition according to the invention can be for use before and/or during the weaning period.

The composition of the present invention can be in solid (e.g. powder), liquid or gelatinous form.

Since infants born by C-section are especially targeted, the composition could advantageously be a nutritional composition consumed in liquid form. It may be a nutritionally complete formula such as an infant formula, a starter formula, a follow-on formula or a fortifier such as a human milk fortifier.

The composition according to the invention generally also contains a protein source, preferably in an amount below 2.0 g per 100 kcal, even more preferably in an amount below 1.8 g per 100 kcal. The type of protein is not believed to be critical to the present invention provided that the minimum requirements for essential amino acid content are met and satisfactory growth is ensured. Thus, protein sources based on whey, casein and mixtures thereof may be used as well as protein sources based on soy. As far as whey proteins are concerned, the protein source may be based on acid whey or sweet whey or mixtures thereof and may include alpha-lactalbumin and beta-lactoglobulin in any desired proportions.

The proteins may be intact or hydrolysed or a mixture of intact and hydrolysed proteins. By the term “intact” is meant that the main part of the proteins are intact, i.e. the molecular structure is not altered, for example at least 80% of the proteins are not altered, such as at least 85% of the proteins are not altered, preferably at least 90% of the proteins are not altered, even more preferably at least 95% of the proteins are not altered, such as at least 98% of the proteins are not altered. In a particular embodiment, 100% of the proteins are not altered.

The term “hydrolysed” means in the context of the present invention a protein which has been hydrolysed or broken down into its component peptides or amino acids.

The proteins may be either fully or partially hydrolysed. It may be desirable to supply partially hydrolysed proteins (degree of hydrolysis between 2 and 20%), for example for infants and young children believed to be at risk of developing cow's milk allergy. If hydrolysed proteins are required, the hydrolysis process may be carried out as desired and as is known in the art. For example, a whey protein hydrolysate may be prepared by enzymatically hydrolysing the whey fraction in one or more steps. If the whey fraction used as the starting material is substantially lactose free, it is found that the protein suffers much less lysine blockage during the hydrolysis process. This enables the extent of lysine blockage to be reduced from about 15% by weight of total lysine to less than about 10% by weight of lysine; for example about 7% by weight of lysine which greatly improves the nutritional quality of the protein source. In an embodiment of the invention at least 70% of the proteins are hydrolysed, preferably at least 80% of the proteins are hydrolysed, such as at least 85% of the proteins are hydrolysed, even more preferably at least 90% of the proteins are hydrolysed, such as at least 95% of the proteins are hydrolysed, particularly at least 98% of the proteins are hydrolysed. In a particular embodiment, 100% of the proteins are hydrolysed.

In a particular embodiment the composition according to the invention is a hypoallergenic composition. In another particular embodiment the composition according to the invention is a hypoallergenic nutritional composition.

The composition according to the present invention generally contains a carbohydrate source. This is particularly preferable in the case where the nutritional composition of the invention is an infant formula. In this case, any carbohydrate source conventionally found in infant formulae such as lactose, saccharose, maltodextrin, starch and mixtures thereof may be used although the preferred source of carbohydrates is lactose.

The composition according to the present invention generally contains a source of lipids. This is particularly relevant if the nutritional composition of the invention is an infant formula. In this case, the lipid source may be any lipid or fat which is suitable for use in infant formulae. Preferred fat sources include palm oleic, high oleic sunflower oil and high oleic safflower oil. The essential fatty acids linoleic and olinolenic acid may also be added, as well small amounts of oils containing high quantities of preformed arachidonic acid and docosahexaenoic acid such as fish oils or microbial oils. The fat source preferably has a ratio of n-6 to n-3 fatty acids of about 5:1 to about 15:1; for example about 8:1 to about 10:1.

The composition of the invention also contains preferably all vitamins and minerals understood to be essential in the daily diet and in nutritionally significant amounts. Minimum requirements have been established for certain vitamins and minerals. Examples of minerals, vitamins and other nutrients optionally present in the composition of the invention include vitamin A, vitamin B1, vitamin B2, vitamin B6, vitamin B12, vitamin E, vitamin K, vitamin C, vitamin D, folic acid, inositol, niacin, biotin, pantothenic acid, choline, calcium, phosphorous, iodine, iron, magnesium, copper, zinc, manganese, chlorine, potassium, sodium, selenium, chromium, molybdenum, taurine, and L-carnitine. Minerals are usually added in salt form. The presence and amounts of specific minerals and other vitamins will vary depending on the intended population. If necessary, the composition of the invention may contain emulsifiers and stabilisers such as soy, lecithin, citric acid esters of mono- and di-glycerides, and the like.

The composition of the invention may also contain other substances which may have a beneficial effect such as nucleotides, nucleosides, and the like.

The composition according to the invention may be prepared in any suitable manner. A composition will now be described by way of example.

For example, a formula such as an infant formula may be prepared by blending together the protein source, the carbohydrate source and the fat source in appropriate proportions. If used, the emulsifiers may be included at this point. The vitamins and minerals may be added at this point but they are usually added later to avoid thermal degradation. Any lipophilic vitamins, emulsifiers and the like may be dissolved into the fat source prior to blending. Water, preferably water which has been subjected to reverse osmosis, may then be mixed in to form a liquid mixture. The temperature of the water is conveniently in the range between about 50° C. and about 80° C. to aid dispersal of the ingredients. Commercially available liquefiers may be used to form the liquid mixture.

If the final product is to be a powder, they may likewise be added at this stage if desired. The liquid mixture is then homogenised, for example in two stages.

The liquid mixture may then be thermally treated to reduce bacterial loads, by rapidly heating the liquid mixture to a temperature in the range between about 80° C. and about 150° C. for a duration between about 5 seconds and about 5 minutes, for example. This may be carried out by means of steam injection, an autoclave or a heat exchanger, for example a plate heat exchanger.

Then, the liquid mixture may be cooled to between about 60° C. and about 85° C. for example by flash cooling. The liquid mixture may then be again homogenised, for example in two stages between about 10 MPa and about 30 MPa in the first stage and between about 2 MPa and about 10 MPa in the second stage. The homogenised mixture may then be further cooled to add any heat sensitive components, such as vitamins and minerals. The pH and solids content of the homogenised mixture are conveniently adjusted at this point.

If the final product is to be a powder, the homogenised mixture is transferred to a suitable drying apparatus such as a spray dryer or freeze dryer and converted to powder. The powder should have a moisture content of less than about 5% by weight. The human milk oligosaccharide(s) and/or the precursor(s) thereof may be added at this stage by dry-mixing or by blending them in a syrup form of crystals, along with the probiotic strain(s) (if used), and the mixture is spray-dried or freeze-dried.

If a liquid composition is preferred, the homogenised mixture may be sterilised then aseptically filled into suitable containers or may be first filled into the containers and then retorted.

### Use in Treatment and Prevention

The complex coacervates of the present invention or the composition of the present invention can advantageously be used in therapy. Thus the invention also provides for such complex coacervates and such composition for use in therapy or prevention. In a particularly preferred aspect the invention provides for complex coacervates of the present invention and compositions comprising such complex coacervates for use in therapy or prevention.

Therapy is intended here as the curing or prevention of a disease or malfunction of the body and also covers prophylactic treatment, i.e., prevention of an adverse medical condition. Therapy is also intended here to include human and animal therapy. In other words, the present invention relates to a method for treating a subject comprising administering to the subject a therapeutically effective amount of a complex or of a product according to the present invention.

The present invention therefore relates to a complex, preferably a complex coacervate, comprising lactoferrin and osteopontin or a composition comprising a complex, preferably a complex coacervate, comprising lactoferrin and osteopontin for use in the treatment or prevention of metabolic disorders, in particular overweightness, obesity, pre-diabetes or diabetes, and/or inflammatory diseases, in particular sepsis or necrotizing enterocolitis.

The present invention therefore also relates to a method for treating or preventing metabolic disorders, in particular overweightness, obesity, pre-diabetes or diabetes, and/or inflammatory diseases, in particular sepsis or necrotizing enterocolitis, by administering a complex, preferably a complex coacervate, comprising lactoferrin and osteopontin or a composition comprising a complex, preferably a complex coacervate, comprising lactoferrin and osteopontin to a subject.

The definitions and embodiments for the complex coacervates and the composition comprising the complex coacervates of the present invention as described herein-above apply mutatis mutandis to their use in therapy and prevention.

The complexes, in particular the complex coacervates, and the composition comprising the complexes, in particular the complex coacervates, are preferably administered to a subject in need thereof.

In a particular embodiment, the subject is an infant, a young child or toddler. In a particular embodiment, the subject is an infant. In a particular embodiment, the subject is an infant which has been born in term or pre-termed, in particular by C-section.

The complex coacervates according to the present invention are particularly beneficial as they provide lactoferrin with a high bioactive to subject even after gastrointestinal digestion. Thereby, the complex coacervates according to the present invention provide lactoferrin to a subject which is bioactive after digestion so that the therapeutic and/or preventive effect of lactoferrin and/or osteopontin can be provided even after gastrointestinal digestion.

The complex coacervate (e.g. in the form of a nutritional composition, supplement etc.) may be administered by any suitable route, for example by oral, enteral, or parenteral administration. In preferred embodiments, the complex coacervate is administered orally.

The complex coacervate (e.g. in the form of a nutritional composition, supplement etc.) may be administered in any suitable dose, e.g. to provide a therapeutically effective amount of the complex coacervate. The complex coacervate may be administered in a dose of at least 100 mg/kg/day, at least 200 mg/kg/day, at least 300 mg/kg/day, at least 400 mg/kg/day, at least 500 mg/kg/day, or at least 1000 mg/kg/day. The complex coacervate may be administered in a dose of 10000 mg/kg/day or less, 5000 mg/kg/day or less, 4000 mg/kg/day or less, 3000 mg/kg/day or less, 2000 mg/kg/day or less. In some embodiments, the complex coacervate is administered in a dose of 100 to 10000 mg/kg/day, preferably in a dose of 500 to 5000 mg/kg/day and more preferably in a dose of 1000 to 2000 mg/kg/day.

Use in Promoting Bone Metabolism and/or Homeostasis

The complex coacervate may promote normal bone metabolism and homeostasis when administered to a subject. Studies have shown that lactoferrin and osteopontin play a role in bone metabolism and homeostasis. (see e.g. Naot, D., et al., 2005. Clinical Medicine & Research, 3(2), pp. 93-101; and Si, J., et al., 2020. Medical science monitor: international medical journal of experimental and clinical research, 26, pp. e919159-1).

Thus, the invention also provides for a complex coacervate comprising lactoferrin and osteopontin for use in promoting bone development, growth, strength and/or healing, or preventing and/or treating a bone disease.

The present invention also provides a method of promoting bone development, growth, strength and/or healing, or preventing and/or treating a bone disease, the method comprising administering to a subject in need thereof a therapeutically effective amount of a complex coacervate comprising lactoferrin and osteopontin.

The present invention also provides use of a complex coacervate comprising lactoferrin and osteopontin for the manufacture of a medicament for promoting bone development, growth, strength and/or healing, or preventing and/or treating a bone disease.

The invention provides for a complex coacervate comprising lactoferrin and osteopontin for use in promoting bone development. As used herein, “promoting bone development” may refer to the support of normal bone metabolism, for example during childhood and adolescence, and/or homeostasis. During childhood and adolescence bones are sculpted by a process called modelling, which allows for the formation of new bone at one site and the removal of old bone from another site within the same bone. Bone remodelling is a lifelong process where mature bone tissue is removed from the skeleton and new bone tissue is formed. Supporting normal bone metabolism and/or homeostasis may refer to support of bone normal modelling and/or remodelling (see e.g. Allen, M. R. and Burr, D. B., 2014. Bone modeling and remodeling. In Basic and applied bone biology (pp. 75-90). Academic Press). Supporting normal bone metabolism and/or homeostasis may result in normal bone anatomy and physiology (Clarke, B., 2008. Clinical journal of the American Society of Nephrology, 3(Supplement 3), pp. S131-S139).

The invention provides for a complex coacervate comprising lactoferrin and osteopontin for use in promoting bone growth and/or strength. As used herein, “promoting bone growth and/or strength” may refer to the support of normal bone growth and/or strength, for example during childhood and adolescence. Supporting normal bone growth and/or strength may result in normal bone anatomy and physiology. Suitable methods and parameters to determine bone growth and bone strength will be known to the skilled person (see e.g. Donnelly, E., 2011. Clinical Orthopaedics and Related Research, 469(8), pp. 2128-2138). Suitably, normal bone growth and/or strength may be determined using one or more bone parameter selected from: trabecular bone volume and tissue volume fraction (BV/TV), bone mineral density (BMD), bone mineral content (BMC), cortical bone volume (Ct.BV), medio-lateral diameter, antero-posterior diameter, bone force yield, and bone stiffness. Suitable methods to determine these parameters will be available to the skilled person.

The invention provides for a complex coacervate comprising lactoferrin and osteopontin for use in promoting bone healing. As used herein, “promoting bone healing” may refer to the support of normal bone healing, for example following fractures. Fractures are one of the most frequent injuries of the musculoskeletal system. Although fracture treatment has improved considerably in recent decades, a large proportion of all fractures still display delayed healing and complications including non-union (Claes, L., et al., 2012. Nature Reviews Rheumatology, 8(3), pp. 133-143). Thus, supporting normal bone healing may, for example, prevent delayed union and/or non-union. Increasing age may increase the risk of delayed union or non-union. The complex coacervate may prevent and/or reduce the frequency and/or occurrence and/or severity and/or duration of fractures.

The invention provides for a complex coacervate comprising lactoferrin and osteopontin for use in preventing and/or treating a bone disease. As used herein, the term “bone disease” may refer to medical conditions which affect the bone, in particular related to the reduction of bone organic matrix. Suitably, the bone disease may be a metabolic bone disease. As used herein, the term “metabolic bone disease” may refer to bone disorders caused by deficiencies of minerals such as calcium, phosphorus, magnesium or vitamin D (see e.g. Mays, S., 2007. Advances in human palaeopathology, pp. 215-251). Such disorders may include low bone density, osteoporosis, osteopenia, rickets, osteomalacia, Paget's disease of bone, hypophosphatasia, scurvy and osteitis fibrosa cystica. In some embodiments, the bone disease is selected from low bone density, osteopenia, osteoporosis, osteomalacia, and rickets.

Metabolic bone disease is frequent in preterm and/or low birth weight infants and/or infants suffering from suboptimal intra-uterine nutrition and leads to an increased risk of bone fractures in these populations (Arch Dis Child Fetal Neonatal Ed 2002 86: F82-F85). Infants, children and adolescents suffering from growth retardation due to malnutrition and/or disease are also frequently affected by these conditions.

The invention provides for a complex coacervate comprising lactoferrin and osteopontin for use in preventing and/or treating low bone density. Bone density, or bone mineral density (BMD), is the amount of bone mineral in bone tissue and is used in clinical medicine as an indirect indicator of osteoporosis and fracture risk. It is measured by a procedure called densitometry. BMD tests provide individuals with a measurement called a T-score, a number value that results from comparing the bone density of the individuals to optimal bone density. A subject with low bone density may have a bone mineral density that is more than 1.0 standard deviations below the mean peak bone mass (average of young, healthy adults) as measured by DXA (Dual-energy X-ray absorptiometry).

The invention provides for a complex coacervate comprising lactoferrin and osteopontin for use in preventing and/or treating osteopenia. Osteopenia is a condition characterized by deficient organic bone matrix leading to amounts of bone tissue lower than normal. Osteopenia may be defined as a bone mineral density between that is between 1.0 and 2.5 standard deviations below the mean peak bone mass as measured by DXA.

The invention provides for a complex coacervate comprising lactoferrin and osteopontin for use in preventing and/or treating osteoporosis. Osteoporosis (“porous bones”, from Greek) is a disease of bone that leads to an increased risk of fracture. This disease is characterized by too little bone formation, excessive bone loss, or a combination of both. In osteoporosis the bone mineral density (BMD) is reduced, bone microarchitecture is deteriorating, and the amount and variety of proteins in bone is altered. Osteoporosis may be defined as a bone mineral density that is 2.5 standard deviations or more below the mean peak bone mass as measured by DXA.

The invention provides for a complex coacervate comprising lactoferrin and osteopontin for use in preventing and/or treating osteomalacia or rickets. Osteomalacia is a condition where bone mineral density (BMD) and bone mineral content (BMC) is lower than normal. Osteomalacia in children is usually associated to rickets. Osteomalacia or rickets may show signs as diffuse body pains, muscle weakness, and fragility of the bones. The most common cause of the disease is a deficiency in vitamin D, which is normally obtained from the diet and a sunlight exposure.

The complex coacervate may increase bone growth and/or bone strength when administered to a subject. For example, when the complex coacervate is administered to a subject it may increase bone growth and/or bone strength compared to a subject who is not administered the complex coacervate, or who is administered lactoferrin and osteopontin in a different form (e.g. as a blend or soluble complex). Suitably, the complex coacervate increases bone growth and/or bone strength when compared to the same dose of lactoferrin and osteopontin provided as a soluble complex. Suitably, the complex coacervate increases bone growth and/or bone strength when compared to the same dose of lactoferrin and osteopontin provided as a blend. Suitably, the complex coacervate increases one or more bone parameter selected from: trabecular bone volume and tissue volume fraction (BV/TV), bone mineral density (BMD), bone mineral content (BMC), cortical bone volume (Ct.BV), medio-lateral diameter, antero-posterior diameter, bone force yield, and bone stiffness. Suitable methods to determine these parameters will be available to the skilled person.

The subject may be any suitable subject. Suitably, the subject may be a mammal. In preferred embodiments, the subject is a human. In other embodiments, the subject is an animal, preferably wherein the animal is a pet. A pet may be an animal selected from dogs, cats, birds, fish, rodents such as mice, rats, and guinea pigs, rabbits, etc.

The present invention is particularly suitable for infants and young children at risk of bone disease, having a family history of bone disease, or having already experienced at least one, preferably several, episode(s) of fracture. The present invention is also particularly suitable for infants and young children who were born preterm or with low-birth weight or experienced intra-uterine growth retardation or who suffered from growth stunting because of malnutrition or experienced disease such as Crohn's disease and/or celiac disease and/or cancer or who were treated with drugs leading to malabsorption, anorexia and/or metabolic bone disease, such as chemotherapy drugs and/or corticosteroids. The present invention is particularly preferred for use in infants and children who were born preterm or with low-birth weight or experienced intra-uterine growth retardation, or with intra-uterine malnutrition or who suffered growth delay.

In some embodiments, the subject is a juvenile, an adolescent, a child, or an infant. The term “juvenile” may refer to an individual that has not yet reached adulthood. The term “adolescent” may refer to an individual during the period from the onset of puberty to adulthood. The term “child” may refer an individual between the stages of birth and puberty.

In some embodiments, the subject was born preterm or with low-birth weight or experienced intra-uterine growth retardation. The term “preterm infant” may refer to an infant born at least than 37 weeks gestational age. The term “low birth weight infant” may refer to an infant having a live-born weight less than 2,500 g.

In some embodiments, the subject suffered from stunted growth. The definition of stunting may refer to the “height for age” value to be less than two standard deviations of the WHO Child Growth Standards median (see e.g. De Onis, M. and Branca, F., 2016. Maternal & child nutrition, 12, pp. 12-26). In some embodiments, the subject suffered from growth stunting because of malnutrition or experienced disease such as anorexia, Crohn's disease and/or celiac disease. In some embodiments, the subject suffered from growth stunting because of treatment with drugs leading to malabsorption, anorexia and/or metabolic bone disease, such as chemotherapy drugs and/or corticosteroids.

The present invention can also apply to adolescents or adults at risk of bone disease or having experienced at least one, preferably several, episode(s) of fractures, or who were born preterm or with low-birth weight or experienced intra-uterine growth retardation of who suffered from growth stunting because of malnutrition or experienced disease such as Crohn's disease and/or celiac disease and/or cancer or who were treated with drugs leading to malabsorption, anorexia and/or metabolic bone disease, such as chemotherapy drugs and/or corticosteroids or who suffered from growth delays because of disease or malnutrition or drugs' use during infancy and/or childhood (including adolescence).

In some embodiments, the subject is an adolescent or an adult. In some embodiments, the subject is an adult, preferably wherein the subject is elderly. In some embodiments, the subject is at least 60 years of age, at least 65 years of age, at least 70 years of age, at least 75 years of age, or at least 80 years of age. The subject may have one or more fractures. The subject may have or may be at risk of one or more delayed union and/or one or more non-union.

### EXAMPLES

f

### 1. Materials

The lactoferrin (LF) powder has a total protein, ash and moisture content of 98.7% (w/w, Dumas nitrogen ×6.25), 0.37% (w/w) and 0.42% (w/w), respectively, as determined according to official AOAC methods (AOAC, 2005). The purity of LF is 95% of total protein (w/w, by HPLC at 214 nm). The spray-dried bovine osteopontin (OPN) powder used has a total protein, moisture and ash content of 89.6% (w/w, Dumas nitrogen ×7.17), 2.22% (w/w), 9.2% (w/w), respectively. The purity of OPN is 99% of total protein as per supplier specification.

### 2. Preparation of Complex Coacervates

Individual protein solutions of LF and OPN were prepared by hydrating the respective powders in ultrapure water (18.2 MΩ·cm) at 25° C. with magnetic stirring for ˜2 h, followed by magnetic stirring overnight (˜18 h) at 4° C. to ensure complete rehydration.

The individual protein solutions of LF and OPN were mixed in 50 mL polypropylene centrifuge tubes. Following pH adjustment, these solutions were centrifuged using a Sorvall RC 5C Plus centrifuge with a Sorvall GSA rotor at a relative centrifugal force (RCF) of 3007 for 20 min at 20° C., to accelerate phase separation. For the remainder of experiments, the mixed solutions were given time to settle naturally under quiescent conditions for ˜18 h at 4° C.

In samples where phase separation occurred, mass balance determination was performed by isolating phases using a micropipette prior to mass measurement using a 4 point analytical balance.

Complex coacervates of lactoferrin and osteopontin are formed at a pH 4 at a LF:OPN mixing ratio (mass protein basis) of 2, at a pH of 5 at LF:OPN mixing ratios (mass protein basis) 4 and 6 and a pH of 6 at a LF:OPN mixing ratio (mass protein basis) of 8.

Complex Coacervates of lactoferrin and osteopontin were formed at the highest yield of coacervation at a pH of 5 and a LF:OPN mixing ratio (mass protein basis) of 4. Maximum coacervate yield refers to the mixed solution whereby the greatest % of total nitrogen resides in the coacervate phase or where the lowest % of total nitrogen resided in the supernatant phase such as for example as soluble complexes.

### 3. Measurement of the Structure of the Complex Coacervates

The microscopic appearance of complex coacervates of LF and OPN was imaged using a Leica DM1000 Optical Microscope (Leica Microsystems GmbH, DE) equipped with a LabCam® smartphone adaptor (iDu Optics, USA) to facilitate digital image capturing. The microscope was operated at a magnification of 40× in both phase contrast and darkfield modes of operation. To prepare the samples for imaging, the complex coacervates of LF and OPN (prepared at LF:OPN mass mixing ratio of 4:1, pH 5, 5% w/v protein) were first isolated and dispersed in water after which ˜20 μL of sample was placed between a microscope slide and coverslip. Samples were imaged at least 3 times to ensure representative images were captured.

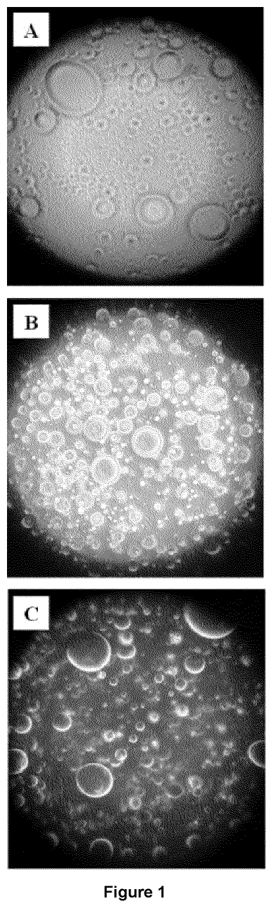

FIG. 1 shows that micron-scale LF-OPN co-precipitates and/or self-aggregates of either protein did not co-exist with the complex coacervates, as all visible entities appeared spherical (liquid) in nature without the presence of irregular-shaped (solid) flocs.

### 4. Measurement of Heat Stability of the Complex Coacervates

The impact of complex coacervation of lactoferrin and osteopontin over lactoferrin and osteopontin is shown with regard to the heat stability of lactoferrin in the complex coacervate over lactoferrin alone by means of differential scanning calorimetry analysis.

Differential scanning calorimetry analysis was performed using a T2500 Discovery Differential Scanning Calorimeter (TA Instruments, Crawley, UK). About 75 μL of sample (6%, w/v, protein) was transferred to high-volume stainless-steel pans which were then sealed using a T_{zero} press (TA Instruments, Crawley, UK). A similar volume of ultrapure water was used as a reference. Samples were equilibrated at 20° C. prior to heating from 20 to 100° C. at 1° C./min followed by cooling at 10° C./min to 20° C. Measurements were performed in triplicate and the heat flow curves were processed using Trios 8.32 software.

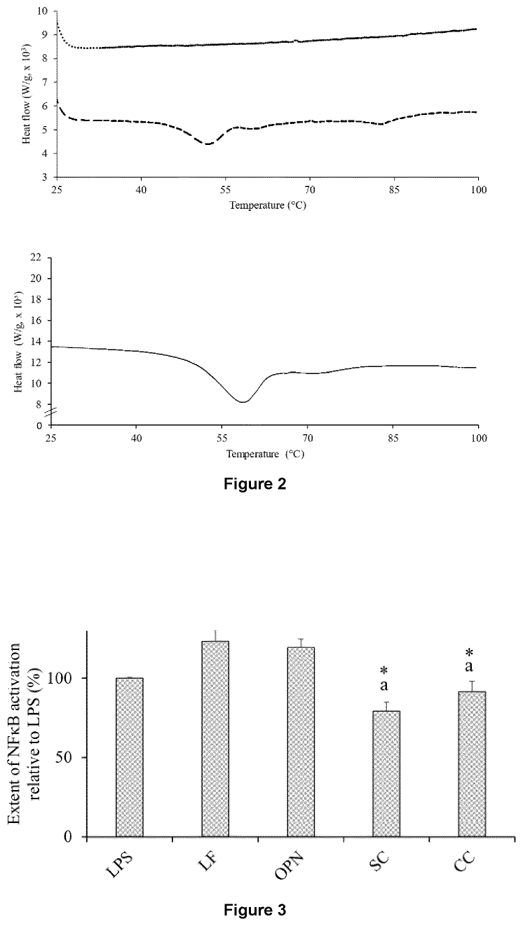

FIG. 2 shows the differential scanning calorimetry thermograms of 5% w/v solutions of lactoferrin (dashed line) and osteopontin (dotted line) at pH 5.0 (FIG. 1, above) and LF/OPN complex coacervate at pH 5.0 (prepared at protein mass ratio 4:1, 5% w/v protein) which had a protein concentration of 27.4% (w/w).

FIG. 2 thereby shows that the LF/OPN complex coacervate has a different heat capacity to that of the individual components and causes a shift in the Tₘ of individual, un-complexed protein peaks.

The thermogram in FIG. 2 therefore confirms the formation of complex coacervates between LF and OPN, which resulted in improved heat stability of LF.

5. Measurement of the Bioactivity after Gastrointestinal Digestion

### 5.1. Preparation of Comparative Meals and Meals According to the Invention

Meals undergoing simulated infant gastrointestinal digestion involving combinations of infant formula (IF) and protein powders were dry mixed prior to hydration.

Combinations prepared were: IF (having a protein content of 10.46% w/w, fat 28.7% w/w, carbohydrate 53.8% w/w, moisture 2.2% w/w and ash 2.16% w/w) with additions of (1) LF to achieve 600 mg/L when hydrated, (2) OPN to achieve 150 mg/L, and (3) soluble complex of LF and OPN or (4) complex coacervate of LF and OPN to achieve 600 mg LF/L.

Meals were prepared by hydrating the relevant powders in ultrapure water (18.2 MΩ·cm) to a total protein concentration of 1.34% (w/v). All meals had the same protein concentration to ensure the enzyme:substrate ratio was consistent between samples for simulated digestions. The protein concentration was chosen based on the recommended reconstitution rate for the model formula used.

### 5.2. Simulated Infant Gastrointestinal Digestion

Simulated infant gastrointestinal digestions were performed using the static in vitro method proposed by Ménard et al. (O. Ménard et al., Food, Chemistry, 2018, 240, 338-345). This model is based on physiological findings in term infants, all parameter justifications are available at Ménard et al.,

Digestions were performed in 40 mL conical, screw capped, amber glass vials which were soaked in 6% (v/v) nitric acid before each digestion to remove residues. The vials were placed in water bath at 37° C. and agitated at 50 rpm for the duration of the digestion process (1 h gastric and 1 h intestinal).

Three independent trials were performed for gastric (pH 5.3) and gastrointestinal (intestinal stage pH 6.6) digestions of each sample. The gastric lipase (19 U/mL) and pepsin activities (268 U/mL) were achieved using rabbit gastric extract (RGE70, Lipolytech, France) and porcine pepsin (Sigma P6887).

Continuous timepoint samples were collected which were instantaneously inactivated by one of three techniques depending on the end point analysis (described in respective sections).

### 5.3. Cell Culture

HT-29 clone 34 cells were used. This cell line permits the expression of a reporter gene for secreted alkaline phosphatase (SEAP) following activation of the NF-κB signaling pathway, as described in Goulding et al., Food Chemistry, 362, 130142. The HT-29 clone 34 cells were cultured in Dulbecco's modified eagle medium (DMEM) supplemented with 10% (v/v) heat-inactivated fetal bovine serum (FBS) and 1% (v/v) non-essential amino acids. Cells were maintained in T75 culture flasks in a 37° C. incubator at 5% CO2.

### 5.4. Cell Culture Experiments

HT-29 clone 34 cells were cultured as described above. For experiments, cells were seeded at 2×10⁵ cells/mL in 24-well format cell culture plates (Greiner Bio-One, Austria) and cultured for 3 d prior to treatment to ensure monolayer confluence. For cell treatment, media was removed, treatments were prepared in fresh media and added to wells to a final well volume of 1 mL. The treatments applied to cells were either undigested or digested forms of the relevant meals. All treatments were filtered using 0.45 μm sterile filters prior to cell administration. Gastric digestates collected for cell culture analysis were preserved by raising the pH above 7 using a 0.1 M phosphate buffer (to inhibit pepsin activity) before freezing, while gastrointestinal digestates were preserved by addition of Pefabloc® (to inhibit serine protease activity) to a final concentration of 1 mM before freezing. Human milk serum (HMS) was added at a concentration of 5% (v/v) of the well volume to provide a source of effector molecules, including soluble CD14 (sCD14). The HMS used was prepared as previously described in Goulding et al., Food Chemistry, 362, 130142. The sCD14 concentration of the HMS was 48 μg/mL. Ethical approval and prior informed consent for the use of the donor human milk samples for research purposes was obtained for the samples in this study. Ethical approval was granted by the Clinical Research Ethics Committee of the Cork Teaching Hospitals, Cork, Ireland. The lipopolysaccharide (LPS) used in the experiments was of the gram-negative Escherichia coli O111:B4 serotype (Sigma, L2630). LPS was added to all wells except negative controls at a concentration of 20 ng/mL. Upon administration of treatments, cells were incubated for 16 h at 37° C. and 5% CO2 prior to recovery of cell supernatants and subsequent analysis. Phosphatase activity of SEAP was quantified by bioluminescence using Phosphalight kits (Applied Biosystems, MA, USA) as a marker of NF-κB activation. Bioluminescence was quantified as relative luminescence units (RLU) using a Varioskan Flash multiwell plate reader (Thermo Scientific, MA, USA) at an integration time of 1000 ms. For graphical representation of data, the LPS treatment was set as 100 relative luminescence units and all other data were normalized relative to this.

### 5.5. Behaviour of Undigested and Digested Lactoferrin, Osteopontin, and Complexes or Complex Coacervates Thereof, in a Model of Intestinal Cell Inflammation

The impact of adding gastrointestinal digestates of LF, OPN, SC, and CC to a model of intestinal cell inflammation is presented in FIG. 3. The NF-κB transcription factor was activated when the cells were stimulated with lipopolysaccharide (LPS, 20 ng/mL) of the Escherichia coli O111:B4 serotype. LF, OPN, and all combinations thereof, resulted in a statistically significant inhibition of LPS-induced NF-κB activation when in an undigested, intact form at 0.35 mg protein/mL.

When the intestinal cells were treated with the gastrointestinal digestates, LF and OPN digestates did not inhibit LPS-induced NF-κB activation while the SC and CC digestates both caused a statistically significant inhibition. The inhibition of LPS-induced NF-κB activation by gastrointestinal digestates of SC and CC were not statistically significant from each other but were when compared to LF alone.

FIG. 3 shows the impact of adding gastrointestinal digestates of lactoferrin (LF), osteopontin (OPN), LF-OPN soluble complexes (SC) or LF-OPN complex coacervates (CC) to a model of intestinal cell inflammation. All samples are added at 0.35 mg protein equivalent/mL. The inflammatory model used is Escherichia coli O111:B4 lipopolysaccharide (LPS)-induced NF-κB activation in HT-29 clone 34 cells. LPS (20 ng/mL) and human milk serum (5% v/v) are added to all wells. All data are normalized to give 100 relative luminescence units (RLU) for the LPS treatment and represent mean values±standard error of triplicate measurements from three independent experiments. ‘*’ represents statistically significant differences (P<0.05) from the 100% RLU treatment.

FIG. 3 shows that the ability of LF to inhibit LPS-induced NF-κB activation requires that LF is not completely proteolyzed. It also shows that digesting a pre-complexed or pre-coacervated LF-OPN powder resulted in an altered gastrointestinal digestate bioactivity in this model of intestinal cell inflammation.

### 6. The Lactoferrin-Osteopontin Coacervate Complex and Bone Development, Growth and Strength

C57/bl6 wild type mouse were supplemented orally between post-natal day 2 and 28 with three different osteopontin-lactoferrin mixes (soluble, blend or co-acervate complex, n=10 per group):
Bovine Lactoferrin-Osteopontin soluble complex (SOLUBLE) solution, 1250 mg/kg/day at 20% w/v in distilled water, N=8.
Bovine Lactoferrin-Osteopontin coacervate complex (COACERVATE) solution, 1250 mg/kg/day at 20% w/v in distilled water, N=9.
Bovine Lactoferrin-Osteopontin blend (BLEND) solution, 1000 mg/kg/day of bovine lactoferrin powder+250 mg/kg/day of bovine osteopontin powder at 20% w/v in distilled water, N=9

From days 28 to 170 all mice received the same amount of a standard diet. At the end of the study, femurs were collected to evaluate bone microstructure parameters.

Micro-computed tomography (μCT UCT35, Scanco Medical AG, Basserdorf Switzerland) was used to assess trabecular and cortical microstructure respectively investigated at distal metaphysis and midshaft diaphysis femur as previously described in the literature (Bonnet N, et al. J Bone Miner Res. 2017; doi: 10:1002). Briefly, trabecular and cortical bone regions were evaluated using isotropic 10 um voxels. For the femur trabecular region, to eliminate the primary spongiosa, 100 slices of primary spongiosa taken from the 100 slices of secondary spongiosa under the distal growth plate were analysed. Femur cortical structure was assessed using 50 continuous CT slides located at the femur midshaft. Morphometric variables were computed from binarized images using direct, three-dimensional techniques that do not rely on prior assumptions about the underlying structure (Bonnet N, et al. Med Phys 2009; 36(4):1286-97).

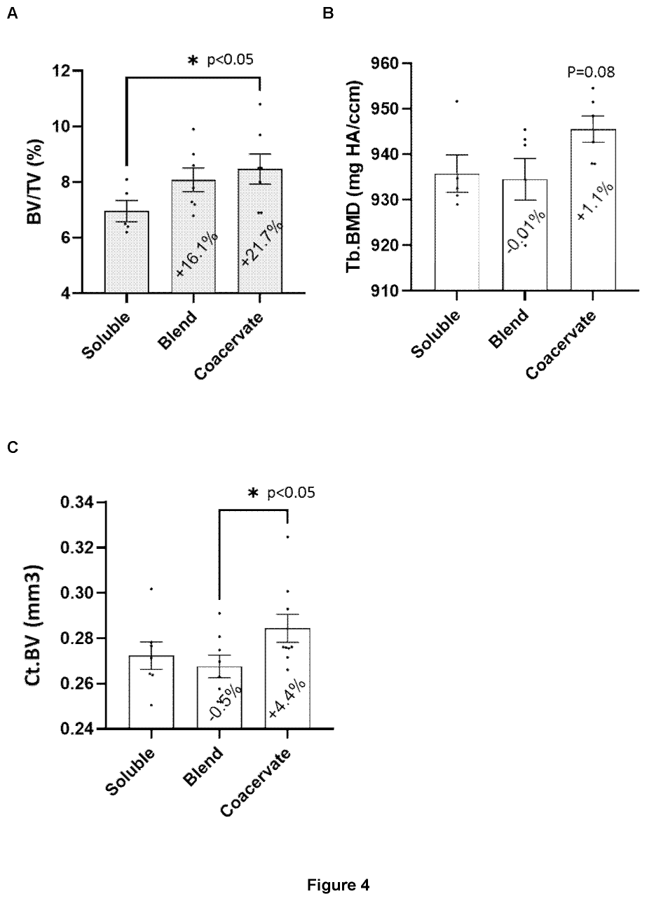

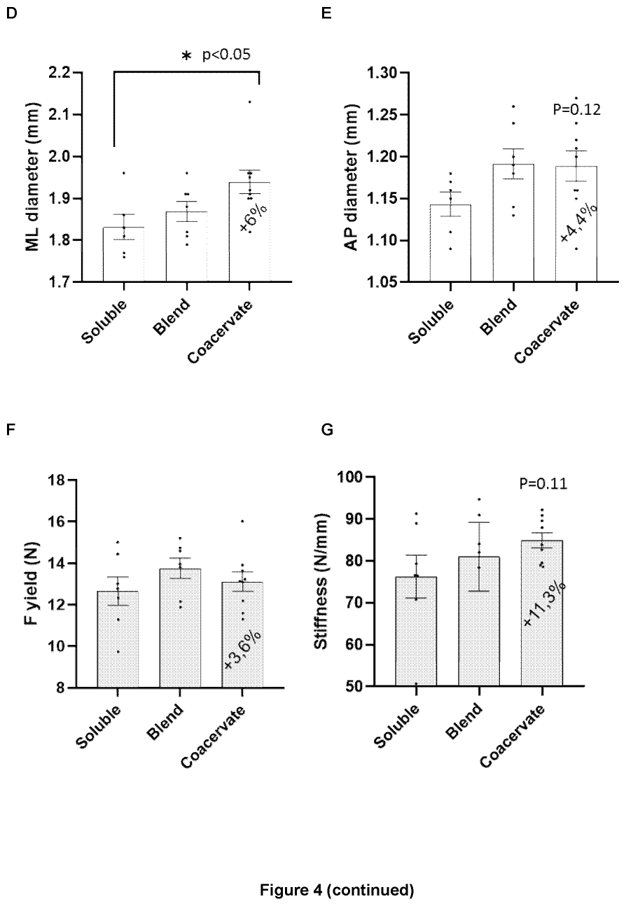

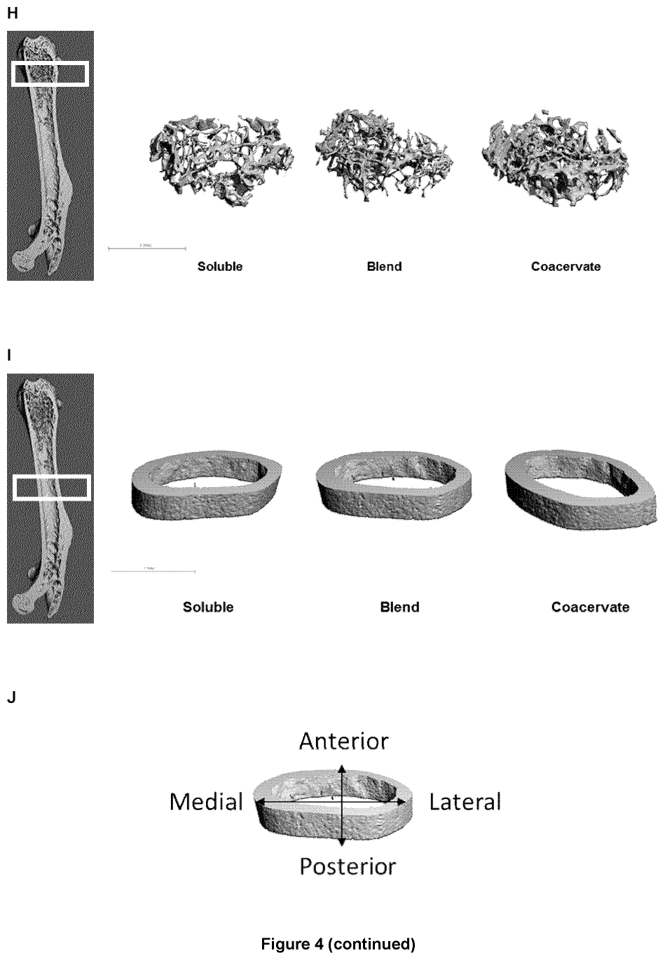

Bone is composed of cortical (or compact) bone and trabecular (or spongy) bone. Cortical bone accounts for approximately 80% of the mass of bone of the human body, and has a lower surface area than trabecular bone due to its lower porosity. Trabecular bone is located at the end of long bones and accounts for approximately 20% of the total mass of the skeleton. Exemplary trabecular and cortical structures are shown in FIGS. 4H and 4I, respectively.

For the trabecular bone regions, the bone volume and tissue volume fraction (BV/TV) and trabecular bone mineral density (Tb.BMD, mg HA/ccm) were assessed. The results are shown in FIGS. 4A and 4B, respectively. For cortical bone at the femoral midshaft, the cortical bone volume (Ct.BV, mm³), antero-posterior and medio-lateral diameters were measured (see FIG. 4J). The results are shown in FIGS. 4C, 4D, and 4E, respectively.

In order to test the biomechanical properties of the bone we performed a three-point bending test, as previously described (Turner C H, Burr D B. Bone 1993; 14:595-608). The load was applied in a compression mode at a nominal deformation rate of 2 mm/minute until fracture. Load-displacement curves were recorded during testing. The force yield and stiffness results are shown in FIGS. 4F and 4G, respectively.

Overall, the results show that mice which were supplemented with the lactoferrin-osteopontin coacervate complex showed improved bone development, growth and strength compared to mice supplemented with different forms of lactoferrin-osteopontin. For example, mice which were supplemented with the lactoferrin-osteopontin coacervate complex had significantly higher trabecular BV/TV, trabecular bone mineral density, and cortical bone volume.

### Embodiments

Various preferred features and embodiments of the present invention will now be described with reference to the following numbered paragraphs (paras).
1. A complex coacervate comprising lactoferrin and osteopontin.
2. The complex coacervate according to para 1, wherein the complex coacervate comprise protein in an amount of 5 to 50% w/w, preferably in an amount of 15 to 40% w/w and more preferably in an amount of 25 to 35% w/w.
3. The complex coacervate according to any one of paras 1 and 2, wherein the complex coacervate comprises water in an amount of 50 to 95% w/w, preferably in an amount of 60 to 85% w/w and even more preferably in an amount of 65 to 75% w/w.
4. The complex coacervates according to any of the preceding paras, wherein the complex coacervate has a diameter of at least 500 nm in the shortest dimension, preferably of at least 600 nm in the shortest dimension, more preferably at least 700 nm in the shortest dimension and more preferably at least 900 nm in the shortest dimension.
5. The complex coacervates according to any of the preceding paras, wherein the complex coacervate has a zeta potential in the range of −15 and +15 mV, preferably in the range of −10 and +10 mV and more preferably in the range of −8 to +5 mV.
6. A process of producing a complex coacervate according to any of the preceding paras, wherein the process comprises the steps of:
   a. providing individual aqueous solutions comprising lactoferrin and osteopontin,
   b. mixing the individual aqueous solutions comprising lactoferrin and osteopontin at a pH of 4 to 6, preferably at a pH of 4.5 to 5.5, more preferably at a pH of 4.8 to 5.2, even more preferably at a pH of 5 and wherein the individual aqueous solutions comprising lactoferrin and osteopontin are adjusted in that the protein mass ratio of lactoferrin to osteopontin is in the range of 2 to 8, preferably in the range of 3 to 6, more preferably in the range of 3.2 to 5.5, more preferably in the range of 3.5 to 5, even more preferably 3.8 to 4.2 and even more preferably 4.
7. The process according to para 6, wherein in step b. the ionic strength in the mixed aqueous solution is not higher than 30 mM, preferably not higher than 20 mM, more preferably not higher than 10 mM, more preferably not higher than 5 mM and even more preferably not higher than 0.2 mM added salts, preferably added inorganic salts, more preferably added NaCl.
8. The process according to any one of paras 6 and 7, wherein in step b. the individual aqueous solutions comprising lactoferrin and osteopontin are adjusted in that the total protein concentration is in the range of 2 to 8% w/v, preferably in the range of 4 to 6% w/v.
9. A composition comprising the complex coacervate according to any one of paras 1 to 5.
10. The composition according to para 9, wherein the composition is selected from the group consisting of food compositions, pet food compositions, drinks, nutritional formulas or nutraceuticals.
11. The composition according to any one of paras 9 and 10, wherein the composition is an infant formula, a starter infant formula, a follow-on formula, a baby food, an infant cereal composition, a growing-up milk, a fortifier such as a human milk fortifier, or a supplement.
12. A complex, preferably a complex coacervate, comprising lactoferrin and osteopontin or a composition comprising a complex, preferably a complex coacervate, comprising lactoferrin and osteopontin for use in the treatment or prevention of metabolic disorders, in particular overweightness, obesity, pre-diabetes or diabetes, and/or inflammatory diseases, in particular sepsis or necrotizing enterocolitis.
13. A method for treating or preventing metabolic disorders, in particular overweightness, obesity, pre-diabetes or diabetes, and/or inflammatory diseases, in particular sepsis or necrotizing enterocolitis, by administering a complex, preferably a complex coacervate, comprising lactoferrin and osteopontin or a composition comprising a complex, preferably a complex coacervate, comprising lactoferrin and osteopontin to a subject.
14. A complex, preferably a complex coacervate, comprising lactoferrin and osteopontin or a composition comprising a complex, preferably a complex coacervate, comprising lactoferrin and osteopontin for use in promoting bone development, growth, strength and/or healing, or preventing and/or treating a bone disease.
15. A method for promoting bone development, growth, strength and/or healing, or preventing and/or treating a bone disease, by administering a complex, preferably a complex coacervate, comprising lactoferrin and osteopontin or a composition comprising a complex, preferably a complex coacervate, comprising lactoferrin and osteopontin to a subject.

### PCT

### (Original in Electronic Form)

(This sheet is not part of and does not count as a sheet of the international application)

| | | |
|---|---|---|
| 0-1 | Form PCT/RO/134 | |
| | Indications Relating to | |
| | Deposited Microorganism(s) | |
| | or Other Biological Material | |
| | (PCT Rule 13bis) | |
| 0-1-1 | Prepared Using | ePCT-Filing-Embedded |
| | | Version 4.11.002 MT/FOP |
| | | 20230213/1.1 |
| 0-2 | International Application No. | |
| 0-3 | Applicant's or agent's | 18221-WO-PCT |
| | file reference | |
| 1 | The indications made below | |
| | relate to the deposited | |
| | microorganism(s) or other | |
| | biological material referred | |
| | to in the description on: | |
| 1-1 | page | 15 |
| 1-2 | line | 12 |
| 1-3 | Identification of deposit | |
| 1-3-1 | Name of depositary | CNCM Collection nationale de |
| | institution | cultures de micro-organismes |
| 1-3-2 | Address of depositary | Collection nationale de cultures |
| | institution | de micro-organismes (CNCM) |
| | | Institut Pasteur, 25-28 |
| | | rue du Docteur Roux, |
| | | 75724 Paris Cedex 15 |
| | | France |
| 1-3-3 | Date of deposit | 12 Jan. 1999 (12 Jan. 1999) |
| 1-3-4 | Accession Number | CNCM I-2116 |
| 1-4 | Additional Indications | |
| 1-5 | Designated States for Which | All designations |
| | Indications are Made | |
| 1-6 | Separate Furnishing of | |
| | Indications | |
| | These indications will be | |
| | submitted to the | |
| | International Bureau later | |
| 2 | The indications made below | |
| | relate to the deposited | |
| | microorganism(s) or other | |
| | biological material referred | |
| | to in the description on: | |
| 2-1 | page | 15 |
| 2-2 | line | 13 |
| 2-3 | Identification of deposit | |
| 2-3-1 | Name of depositary | CNCM Collection nationale de |
| | institution | cultures de micro-organismes |
| 2-3-2 | Address of depositary | Collection nationale de cultures |
| | institution | de micro-organismes (CNCM) |
| | | Institut Pasteur, 25-28 |
| | | rue du Docteur Roux, |
| | | 75724 Paris Cedex 15 |
| | | France |
| 2-3-3 | Date of deposit | 30 Jun. 1992 (30 Jun. 1992) |
| 2-3-4 | Accession Number | CNCM I-1225 |
| 2-4 | Additional Indications | |
| 2-5 | Designated States for Which | All designations |
| | Indications are Made | |
| 2-6 | Separate Furnishing of | |
| | Indications | |
| | These indications will be | |
| | submitted to the | |
| | International Bureau later | |

1. A method for use in promoting bone development, growth, strength and/or healing in a subject, or preventing and/or treating a bone disease in a subject comprising administering to a subject in need a complex coacervate comprising lactoferrin and osteopontin.

2. The method according to claim 1, wherein the complex coacervate comprises protein in an amount of 5 to 50% w/w.

3. The method according to claim 1, wherein the complex coacervate comprises water in an amount of 50 to 95% w/w.

4. The method according to any claim 1, wherein the complex coacervate has a diameter of at least 500 nm in the shortest dimension.

5. The method according to any claim 1, wherein the complex coacervate has a zeta potential in the range of −15 and +15 mV.

6. The method according to claim 1, wherein the complex coacervate is administered in the form of a composition.

7. The method according to claim 6, wherein the composition is selected from the group consisting of food compositions, pet food compositions, drinks, nutritional formulas and nutraceuticals.

8. The method according to claim 6, wherein the composition is in a form selected from the group consisting of an infant formula, a starter infant formula, a follow-on formula, a baby food, an infant cereal composition, a growing-up milk, a fortifier, and a supplement.

9. The method according to claim 1, wherein the complex coacervate is administered orally.

10. The method according to claim 1, wherein the complex coacervate is administered in a dose of 100 to 10000 mg/kg/day.

11. The method according to claim 1, wherein the complex coacervate increases bone growth and/or bone strength.

12. The method according to claim 1, wherein the bone disease is a metabolic bone disorder.

13. The method according to claim 1, wherein the subject is selected from the group consisting of a juvenile, an adolescent, a child, and an infant.

14. The method according to claim 1, wherein the subject is an adult.

15. The method according to claim 1, wherein the subject is an animal.
